SEQUENCE LISTING (33 sequences)

SEQ ID NO: 1 - DNA, 20 bp, Artificial Sequence
  Probe
ggagaagctggcaggctgct

SEQ ID NO: 2 - DNA, 20 bp, Artificial Sequence
  Probe
ggtgagtgaggcaggctgct

SEQ ID NO: 3 - DNA, 21 bp, Artificial Sequence
  Probe
ctgcagctgctgctgtggcac

SEQ ID NO: 4 - DNA, 16 bp, Artificial Sequence
  Probe
agaagctgtgtgccac

SEQ ID NO: 5 - DNA, 16 bp, Artificial Sequence
  Probe
tgagtgagtgtgccac

SEQ ID NO: 6 - DNA, 20 bp, Artificial Sequence
  Probe
tgtgccacctacaagctgtg

SEQ ID NO: 7 - DNA, 18 bp, Artificial Sequence
  Probe
gagctggtgctgctcgga

SEQ ID NO: 8 - DNA, 18 bp, Artificial Sequence
  Probe
ggacactctctgggcatc

SEQ ID NO: 9 - DNA, 18 bp, Artificial Sequence
  Probe
ggacactctctgggcatc

SEQ ID NO: 10 - DNA, 18 bp, Artificial Sequence
  Probe
gcaggctgcttgagccaa

SEQ ID NO: 11 - DNA, 16 bp, Artificial Sequence
  Probe
agaagctggcaggctg

SEQ ID NO: 12 - DNA, 16 bp, Artificial Sequence
  Probe
tgagtgaggcaggctg

SEQ ID NO: 13 - DNA, 18 bp, Artificial Sequence
  Primer
acccccctgggccctgcc

SEQ ID NO: 14 - DNA, 18 bp, Artificial Sequence
  Primer
ctgctgccagatggtggt

SEQ ID NO: 15 - DNA, 20 bp, Artificial Sequence
  Probe
gagcttcctgctcaagtgct

SEQ ID NO: 16 - DNA, 20 bp, Artificial Sequence
  Probe
agagcttcctgctcaagtgc

SEQ ID NO: 17 - DNA, 20 bp, Artificial Sequence
  Probe
gcaagtgaggaagatccagg

SEQ ID NO: 18 - DNA, 20 bp, Artificial Sequence
  Probe
ccagagcttcctgctcaagt

SEQ ID NO: 19 - DNA, 20 bp, Artificial Sequence
  Probe
caagtgaggaagatccaggg

SEQ ID NO: 20 - DNA, 20 bp, Artificial Sequence
  Probe
ctggtgagtggcaggctgct

SEQ ID NO: 21 - DNA, 20 bp, Artificial Sequence
  Probe
cttttcctctaccaggggct

SEQ ID NO: 22 - DNA, 20 bp, Artificial Sequence
  Probe
catagcggccttttcctcta

SEQ ID NO: 23 - DNA, 20 bp, Artificial Sequence
  Probe
ttttcctctaccaggggctc

SEQ ID NO: 24 - DNA, 20 bp, Artificial Sequence
  Probe
tagcggccttttcctctacc

SEQ ID NO: 25 - DNA, 19 bp, Artificial Sequence
  Probe
cggccttttcctctaccag

SEQ ID NO: 26 - DNA, 525 bp, Homo sapiens
acccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctga

SEQ ID NO: 27 - DNA, 417 bp, Homo sapiens
acccccctgggccctgccagctccctgccccagagcttcctgctcaagtgcttagagcaagtgaggaagatccagggcgatggcgcagcgctccaggagaagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctga

SEQ ID NO: 28 - DNA, 19 bp, Artificial Sequence
  Primer
agagccccatgaagctgat

SEQ ID NO: 29 - DNA, 20 bp, Artificial Sequence
  Primer
gacacctccaggaagctctg

SEQ ID NO: 30 - DNA, 20 bp, Artificial Sequence
  Primer
catcttccaggagcgagacc

SEQ ID NO: 31 - DNA, 20 bp, Artificial Sequence
  Primer
tccaccaccctgttgctgta

SEQ ID NO: 32 - DNA, 18 bp, Artificial Sequence
  Primer
acccccctgggccctgcc

SEQ ID NO: 33 - DNA, 18 bp, Artificial Sequence
  Primer
ctgctgccagatggtggt

(19) United States
(12) Patent Application Publication
(10) Pub. No.: US 2009/0269750 A1
(43) Pub. Date: Oct. 29, 2009

(54) MARKER AND METHOD FOR CANCER DIAGNOSIS

(75) Inventors: Sang Yup Lee, Daejeon (KR); Nae-Choon Yoo, Seoul (KR); So Young Yoo, Seoul (KR); Ki-Chang Keum, Seoul (KR); Won-Min Yoo, Seoul (KR)

Correspondence Address: MOORE & VAN ALLEN PLLC, P.O. BOX 13706, Research Triangle Park, NC, 27709, US

(73) Assignee: MEDIGENES CO., LTD., Seoul (KR)

(21) Appl. No.: 12/161,005

(22) PCT Filed: Jan. 18, 2007
(86) PCT No.: PCT/KR07/00300

§ 371 Date: Nov. 14, 2008

(30) Foreign Application Priority Data
Jan. 20, 2006 (KR) 10-2006-0006279

(51) Int. Cl. C12Q 1/68; C07H 21/00; C40B 40/06

(52) U.S. Cl. 435/6; 536/22.1; 506/16

(57) ABSTRACT

The present invention relates to a diagnostic cancer marker using variation of a granulocyte colony stimulating factor (G-CSF) gene and a method for preparing the same, and more specifically, relates to a method for diagnosing cancer and/or assessing the state of cancer progression using an oligonucleotide having the 3′-terminal end of exon 2 region linked to the 5′-terminal end of exon 4 region of a G-CSF gene as a diagnostic cancer marker. According to the present invention, cancer can be quickly and exactly diagnosed using variation in a G-CSF gene expression.

### TECHNICAL FIELD

The present invention relates to a diagnostic cancer marker using variation in the gene expression of a granulocyte colony stimulating factor (G-CSF) and a method for preparing the same, and more specifically, relates to a method for diagnosing cancer and/or assessing the state of cancer progression using an oligonucleotide having the 3′-terminal end of exon 2 region linked to the 5′-terminal end of exon 4 region of a G-CSF gene as a diagnostic cancer marker.

### BACKGROUND ART

Cancer diagnosis is generally achieved by (1) morphological analysis using microscopes such as an optical microscope or electron microscope, (2) immunohistochemical assays which detect proteins specifically expressed in cancer tissues (Iran, Biomed. J., 3:99, 1999; Lancet, 2:483, 1986), or (3) molecular diagnosis which analyzes abnormal biomolecules found in cancer tissues, such as mutated genes. In comparison with the molecular diagnosis, the morphological and immunohistochemical diagnosis requires much longer time and higher cost. Since the molecular diagnosis has a relatively simple procedure and a short time to yield results, it has become a main subject in developing novel diagnostic methods for cancer. Recently, Health Digit Inc. developed a protein chip system for diagnosing various cancers, and gained on approval for clinical tests from the Chinese State Drug Administration (CSDA) for the first time in the world (www.health-digit.com). However, the protein chip system does not use only one biomarker to diagnose all kinds of cancer, but uses 10 or more proteins as biomarkers.

To effectively apply such diagnostic methods to cancer diagnosis, it is most important to select and use cancer diagnostic markers capable of more accurately and easily detecting cancer incidence. Several genes (Steve, M. et al, J. Clin. Oncology, 20:3165˜3175, 2002; Sridlhar, R. et al., J. Clin. Oncology, 20:1932˜1941, 2002) and proteins (Goessl, et al., Urology, 58:335˜338, 2001; Zhou, et al., Breast Cancer Res. Treat., 66:217˜224, 2001; Korea Pat. Publication No. 2001˜0061173) have been reported as diagnostic cancer markers, and some of them are being clinically used for diagnosis of cancer. Among conventional cancer biomarkers, CEA, BFP, TPA and IAP, which have low organ specificity, have low sensitivity, thus generating false positive data. Also, the biomarkers which have high organ specificity, such as AFP, PIVKA II, Esterase I, CA19-9, CA50, Span-1 antigen, CA15-3 and BCA 225, are useful only for target organs.

Many researchers have attempted to find genes having diagnostic applications, in developing diagnostic cancer marker candidates showing different results according to pathological and physiological condition using microarray technology (Liu, H. X. et al., Nat. Genet., 27:55˜58, 2001; Wilson, C. A. et al., Oncogene, 14:1˜16, 1997; Weissensteiner, T., Nucleic Acids Res., 26:687, 1998; Zolezzi, F. et al., Am. J. Med. Genet., 71:366˜370, 1997; Mottes J. R. and Iverson, L. E., Neuron, 14:613˜623, 1995; Crook, R. et al., Nat. Med., 4:452˜455, 1998; Jiang, Z. H. and Wu, J. Y., Proc. Soc. Exp. Biol. Med., 220:64˜72, 1999).

However, since diagnostic cancer marker candidates found by the above mentioned methods are mostly composed of expressed sequence tag (EST), they are just defined as a characteristic of data and thus it is difficult to select reliable specific candidates and to catch on the very genes from which they are originated. Specifically, the number of genes is known by human genome analysis and it is also known that many isoforms or variants are expressed there from to have biological function and its complexity. Therefore, it has become another big subject for the future to find out that, in which gene and condition variants throughout the whole genome are expressed and what their functions are. These various variants can be a good basis to figure out the correlation between the formation of abnormal variants among them and possibility of causing cancer (Cartegni, L. et al., Nat. Rev. Genet., 3:285˜298, 2002; Schweighoffer, F. et al., Pharmacogenomics, 1:187˜197, 2000; Blencowe, B. J., Treds Biochem. Sci., 25:106˜110, 2000; Cooper, T. A. and Mattox, W., Am. J. Hum. Genet., 61:259˜266, 1997).

The present inventors have also conducted studies for a long time to develop a new diagnostic cancer marker which can diagnose various kinds of cancers, consequently, confirmed that deletion of exon 3 region was specifically shown in tumor cells or tumor tissues during transcription of G-CSF gene, thereby filing an application regarding a method for diagnosing cancer using G-CSF mRNA, cDNA variants fragment or protein as a diagnostic cancer marker (WO 2003/027288 A1). In microarray which uses G-CSF gene fragment as a diagnostic cancer marker of the above application patent, any one or more fragments among exons 1, 2, 4 and 5 DNA fragments of G-CSF gene together with exon 3 DNA fragment of G-CSF gene are used as nucleic acid probes to detect G-CSF gene fragment having deleted exon 3 region among biological samples. This inventive method for diagnosing cancer, by detecting deletion of exon 3 region of G-CSF gene expression is one of the technologies which diagnose cancer using characteristics of gene variants, and is considered to be a useful diagnostic cancer marker candidate, since the variants appear in most cancer.

Meanwhile, most genes including G-CSF gene generally express many isoforms and variants, so, probe fragments fixed on a microarray must have high sensitivity in detecting the deletion of exon 3 region of G-CSF gene. Also, the expression of normal G-CSF or their fragments can exist together with that of mutated G-CSF isoforms in tumor cells or tumor tissues, thus diagnosis for cancer only by detecting the presence of exon 3 region of G-CSF in its gene expression can lead to loss of credibility or low sensitivity and, moreover, it has a problem in assessing the state of cancer progression.

Accordingly, the present inventors have made extensive efforts to develop a new nucleic acid probe for detecting G-CSF gene fragment not having exon 3 region which can satisfy the above requirement or solve the above problem, and as a result, found that it has remarkably increased high sensitivity in cancer diagnosis compared with other probes, when an oligonucleotide containing a nucleic acid sequence having the 3′-terminal end of exon 2 region of G-CSF gene linked to the 5′-terminal end of exon 4 region of G-CSF gene is used as a diagnostic cancer marker, and confirmed that the state of cancer progression can be accurately diagnosed by using an oligonucleotide containing nucleic acid sequence having 3′-terminal end of exon 2 region of G-CSF gene linked to the 5′-terminal end of exon 4 region of G-CSF gene together with an oligonucleotide having sequences of a part or the entire region of exon 3 region of G-CSF gene as a diagnostic cancer marker, thereby completing the present invention.

### SUMMARY OF THE INVENTION

Therefore, the main object of the present invention is to provide an oligonucleotide for diagnosing cancer, essentially containing a nucleic acid sequence of a splice junction site having the 3′-terminal end of exon 2 region linked to the 5′-terminal end of exon 4 region of a granulocyte colony stimulating factor gene.

Another object of the present invention is to provide a diagnostic kit for cancer diagnosis containing the oligonucleotide and a method for diagnosing cancer using the oligonucleotide.

To achieve the above objects, the present invention provides an oligonucleotide for a diagnostic cancer marker, essentially containing a nucleic acid sequence of a splice junction site having the 3′-terminal end of exon 2 region linked to the 5′-terminal end of exon 4 region of a G-CSF gene.

Preferably, the oligonucleotide according to the present invention essentially contains nucleic acid sequences of SEQ ID NOs: 1 or 2.

The present invention also provides a diagnostic kit for cancer diagnosis containing the oligonucleotide.

In the present invention, the diagnostic kit for cancer diagnosis is preferably a diagnostic kit for assessing the state of cancer progression which additionally contains an oligonucleotide essentially containing sequences of a part or the entire region of the exon 3 region of G-CSF gene.

The present invention also provides a method for diagnosing cancer, the method comprising the steps of: (a) obtaining a G-CSF nucleic acid sample from a mammal biological sample; (b) amplifying the obtained G-CSF nucleic acid sample; and (c) detecting oligonucleotide containing a nucleic acid sequence of a splice junction site having the 3′-terminal end of exon 2 region linked to the 5′-terminal end of exon 4 region of a G-CSF gene, in the amplified sample.

In the inventive method, the step (c) preferably contains the step in which simultaneously detects an oligonucleotide containing sequences of a part or the entire region of the exon 3 region together with an oligonucleotide containing a nucleic acid sequence of splice junction site having the 3′-terminal end of exon 2 region linked to the 5′-terminal end of exon 4 region of a G-CSF gene, in the amplified sample.

Other features and embodiments of the present invention will be more fully apparent from the following detailed description and appended claims.

### BRIEF DESCRIPTION OF DRAWINGS

FIG. 1 is a schematic diagram of a process of producing normal protein and variants from human G-CSF gene.

FIG. 2 shows a junction region of an exon 2 region and an exon 3 region which can be formed by two types (type A, type B) of exon 2 region of human G-CSF gene.

FIG. 3 shows positions to which primers used in PCR is attached and expected PCR products according to the positions.

FIG. 4 is a design of DNA chip which consists of probes of each region of amplified G-CSF gene (E2: a probe designed from exon 2 region of a G-CSF gene; E2E3a: a probe designed from a junction site having the 3′-terminal end of exon 2 region linked to the 5′-terminal end of exon 3 region of a type A G-CSF gene; E2E3b: a probe designed from a junction site having the 3′-terminal end of exon 2 region linked to the 5′-terminal end of exon 3 region of a type B G-CSF gene; E3-1, E3-3, E3-4 and E3-6: probes designed from exon 3 region of a G-CSF gene; E2E4a: a probe designed from a junction site having the 3′-terminal end of exon 2 region linked to the 5′-terminal end of exon 4 region of a type A G-CSF gene; E2E4b: a probe designed from a junction site having the 3′-terminal end of exon 2 region linked to the 5′-terminal end of exon 4 region of a type B G-CSF gene; P: a probe constructed to distinguish positions by fluorescent labels as a position marker; N: a negative control (spotting solution).

FIG. 5 shows the results of hybridization using DNA chip of FIG. 4. Red circles show probes showing signals.

FIG. 6 shows the results of hybridization in DNA chip of FIG. 4 according to types of cells and tissues (A: normal human blood, B: lung cancer (A549), C: large intestine cancer (SES-T), D: stomach cancer (1:AGS, 2: YCC-2, 3: Hwang00, E: cervical cancer (1: C33A, 2: HeLa), F: breeding (HT1080), G: breast cancer (MDA-MB-231), H: pancreas cancer (Capan-2), I: liver cancer (SK-Hep1), J: malignant melanoma (SK-Mel), K: leukemia (Jurket cDNA library), L: embryonic kidney (293)). Red circles show signals of probes capable of distinguishing between cancer tissues and normal tissues.

FIG. 7 is a schematic view of a DNA chip which consists of probes designed to detect a splice junction site of G-CSF gene (E2E4: a probe designed from a junction site having the 3′-terminal end of exon 2 region linked to the 5′-terminal end of exon 4 region of two types (type A, type B) of G-CSF gene; E2E3: a probe designed from a junction site having the 3′-terminal end of exon 2 region and the 5′-terminal end of exon 3 region of two types (type A, type B) of G-CSF gene; P: a probe constructed to distinguish positions by fluorescent signals as a position marker; N: a negative control (spotting solution).

FIG. 8 shows the results of hybridization in DNA chip of FIG. 7 according to types of cells and tissues. Red circles show a probe which can specifically show signals in case of cancer.

FIG. 9 is a schematic view of a DNA chip on which probes, designed from each exon region of G-CSF gene to examine diagnosis efficiency of each probe, are fixed.

FIG. 10 is a schematic view showing positions of each probe in G-CSF gene. Probes included in an ellipse among probes for G-CSF gene not having exon 3 are designed from the 3′-terminal end of exon 2 and the 5′-terminal end of exon 4 of splicing variants.

FIG. 11 shows signal intensities of probes according to types of cells and probe candidates showing effectiveness, which can be deduced there from.

FIG. 12 shows the results of hybridization using DNA chip of FIG. 9. Red circles show a probe which can specifically show signals in only cancer.

FIG. 13 shows the results of hybridization using DNA chip of FIG. 9 according to types of cells and tissues. Images of hybridization reactions which is obtained by Scanarray 5000 (A: normal blood (WBC), B: 293 (embryonic cell line), C: SES-N (normal large intestine), D: SES-T (large intestine cancer), E: Colo205 (colon cancer cell line), F: DLD-1 (colon cancer cell line), G: Hwang00 (stomach cancer cell), H: YCC-3 (stomach cancer cell line), J: MDA-MB-231 (breast cancer cell line), K: NCI-H460 (lung cancer cell line), L: Caki-2 (kidney cancer cell line), M: Capan-2 (pancreas cancer cell line), N: SK-Mel2 (malignant melanoma), O: HepG-2 (hepatocellular carcinoma), P: SK-Hep1 (liver cancer cell line)). Red circles show a probe which can specifically show signals in case of cancer.

FIG. 14 shows DNA chips which are prepared by mixing each probe with spotting solution. The part marked with a blue square is a region on which a probe representing cancer is located.

FIG. 15 shows the results of hybridization of products obtained by amplifying nucleotide sequence of human G-CSF gene derived from normal and tumor clinical samples using primers of SEQ ID NO: 32 and SEQ ID NO: 33 according to Example 8 and Example 9 with DNA chip of FIG. 14.

### DETAILED DESCRIPTION OF THE INVENTION, AND PREFERRED EMBODIMENTS

The present invention relates to a method for diagnosing cancer and/or assessing the state of cancer progression, using an oligonucleotide which essentially contains a nucleic acid sequence of a splice junction site having 3′-terminal end of exon 2 region linked to the 5′-terminal end of exon 4 as G-CSF gene variants fragment generated after post-transcription process by genetic analysis method including a microarray. In other words, the present invention relates to a method for diagnosing cancer and/or assessing the state of cancer progression using G-CSF gene variants obtained by deleting an exon 3 region in G-CSF gene to link an exon 2 region and an exon 4 region, as a diagnostic cancer marker.

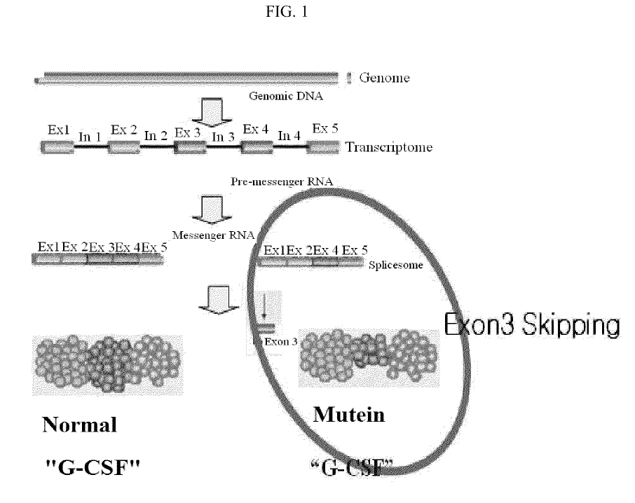

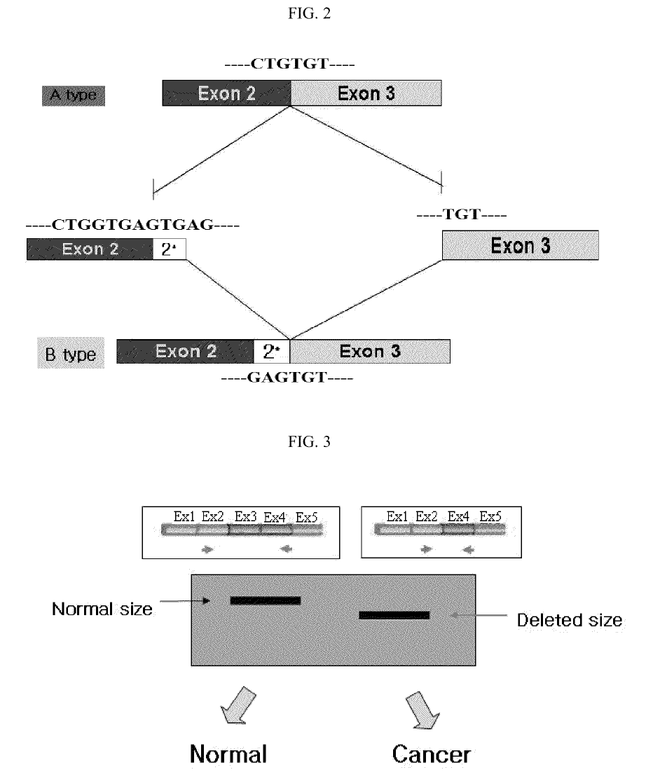

Exons 1˜5 of G-CSF gene are normally linked during splicing process in normal human body, however splicing occurs in a form of a variant not having exon 3 in tumor cells or tumor-progressing cells to produce mRNA not having exon 3 (FIG. 1). Exon 2 region of a human G-CSF gene has two types (type A, type B), therefore, a junction site of exon 2 region and exon 3 region also has two types (FIG. 2). Also, as a result of splicing of G-CSF gene, G-CSF mRNA having all exon 1˜exon 5 is isolated from a normal cell, and mRNA not having exon 3 is isolated from a tumor cell, and the above mentioned difference of mRNAs can be confirmed by PCR using primers specific to G-CSF gene (FIG. 3).

Molecular biological methods which are used in identifying both genes specifically expressed (or suppressed) in tumor cells and genetic mutation are exemplified by PCR (Bottema, C. D., Mutat. Res., 233:93˜102, 1993; Nelson, D. L., Curr. Opin. Genet. Dev., 1:62˜68, 1991; Pourzand, C. and Cerutti, P., Mutat. Res., 288:113˜121, 1993; Holland, P. M. et al., Proc. Natl. Acad. Sci. USA, 8:7276˜7280, 1991), Single-Stranded Conformation Polymorphism (SSCP, Glavac, D., Hum. Mutat., 19:384˜394, 2002; Strippoli, P. et al., Int. J. Mol. Med., 8:567˜572, 2001), DNA Sequencing Analysis (Sanger, F. et al., Proc. Natl. Acad. Sci. USA, 74:6463˜5467, 1997), Protein Truncation Test (Hardy, C. A., Methods Mol. Biol., 187:87˜108, 2002), automatic nucleotide sequence analysis (Boutin, P. et al., Hum. Mutat., 15(2):201˜203, 2000), study of loss of heterozygosity (Yang, Q. et al, Clin. Cancer Res., 8:2890˜2893, 2002), study of microsatellite instability (Furlan, D. et al., J. Pathol., 197:603˜609, 2002), gene analysis using MALDI-TOF (Leushner J, Expert. Rev. Mol. Dign., 1:11 ˜18, 2001), gene analysis by hybridization (Wetmur, J. G., Critical Reviews in Biochem. Mol. Biol., 26:227˜259, 1991), gene analysis using DNA chips (Goessl et al., Urology, 58:335˜338, 2001; Zhou et al., Brest Cancer Res. Treat., 66:217˜224, 2001; Korea Pat. Publication No. 2001˜0061173), analysis using protein chips (Pharmacogenomics, 1:385˜393, 2000). Therefore those skilled in the art will understand that they can easily detect the existence of splice junction site of specific variants according to the present invention, generated in post-transcriptional process of G-CSF by properly using well-known molecular biological methods including the above mentioned methods. The present inventors found that the most effective probes capable of detecting the existence of the variants are only probe candidates capable of detecting the existence of splice junction site, thereby inventing a diagnostic method by which the existence thereof can be detected. However, among the above mentioned methods, the detection of specific variants generated during post-transcriptional process of G-CSF according to the present invention is preferably and easily performed by using PCR, hybridization reaction and DNA chip.

To perform cancer diagnosis according to the present invention, a G-CSF gene or variants thereof should first be obtained from tissue specimens or cells. Since a DNA sample for a specific gene is typically obtained from normal tissues or cells at a very small amount, the specific gene should be amplified by PCR and for such amplification, primers suitable for such amplification should be designed. In the present invention, to amplify a part or an entire region of splice junction site of an exon 2 region and an exon 4 region, DNA nucleic acid fragments to be used as primers in PCR for detecting the existence of the splice junction site is required. That is, the primers, as used herein, refer to oligonucleotides capable of amplifying a nucleotide sequence of G-CSF gene, comprising a part or an entire region of the splice junction site of an exon 2 region and an exon 4 region. Those skilled in the art will be able to easily design such primers. Those skilled in the art will be able to easily design such primers. Therefore, all primers capable of amplifying G-CSF gene variants comprising a part or an entire region of the splice junction site, which can be designed by those skilled in the art, are intended to fall within the scope of the present invention.

In accordance with an aspect of the present invention, there is provided a gene microarray or membrane to which a DNA fragment comprising a splice junction site having the 3′-end of an exon 2 linked to the 5′-end of exon 4 of the G-CSF gene is immobilized, which is useful for diagnosis of cancer. The gene microarray includes DNA chips effective in detection of a gene by hybridization including applying to a complementary oligonucleotide probe immobilized on the surface of a slide glass treated with a specific chemical reagent. Non-limiting examples of the membrane, which can be used instead of the slide glass in hybridization, may include all membranes capable of immobilizing DNA fragments; and preferably, nylon and nitrocellulose membranes.

Fixing the probes on the surface of a slide glass and a membrane can be easily achieved by the conventional technique known in the art. In addition, preparation of targets, hybridization and stripping will be performed according to the conventional techniques common in the art.

In another aspect of the present invention, there is included a composition for diagnosis of cancer, comprising a DNA fragment containing a splice junction site having the 3′-end of an exon 2 linked to the 5′-end of exon 4 of G-CSF gene and a diagnostically acceptable conventional carrier. In a further aspect of the present invention, there is included a diagnostic kit comprising a DNA fragment containing a splice junction site having the 3′-end of an exon 2 linked to the 5′-end of exon 4 of the G-CSF gene and a DNA microarray using the DNA fragment.

### EXAMPLES

Hereinafter, the present invention will be described in more detail by specific examples. However, the present invention is not limited to these examples, and it is obvious to those of ordinary skill in the field of the present invention that numerous variations or modifications could be made within the spirit and scope of the present invention.

### Example 1

### Preparation of Sample from Tissues (Cells)

The normal cell lines and tumor cell lines used in Examples of the present invention are given in Table 1, below. The underlined samples have the same result as those of the normal cell lines in Table 1.

The tumor cell lines listed in Table 1 can be obtained from the cell collection centers listed in Table 1. The tumor cell line, obtained from the cancer metastasis research center at College of Medicine, Yonsei University, was prepared as follows. After ascitic fluid was aseptically obtained from advanced cancer patients, supplemented with heparin in an amount of 10 units per ml to prevent clumping of cells and centrifuged at 400×g for 10 min. The precipitated cells obtained by centrifuge were cultured in a 25 cm² culture flask. In case of containing a large number of erythrocytes, Ficoll-hypaque density gradient centrifugation at 800×g was performed to separate mononuclear cells from erythrocytes, and the obtained mononuclear cell phase was incubated at 37° C. under 5% CO₂. After incubation for 1 day (16˜18 hours), the culture medium was centrifuged at 400×g for 10 min, and the precipitated cells were cultured in a new 25 cm² culture flask. During culturing, cells were observed under a phase contrast microscope, and the culture medium was replaced twice or three times per week. When tumor cell colonies were formed, the tumor cell clusters were obtained by treatment with trypsin-EDTA or by obtaining colony or by using scrapers, or the fluid containing tumor cells was centrifuged to remove normal cells. The resulting pure tumor cells were stored at frozen states according to their passages.

Human leucocyte cells can be obtained as follows. After 8 mL of blood was transferred into 50 mL of Corning tube, 24 mL of RBC lysis buffer was added and the mixture was left to stand at 4° C. for 10 min, while stirring it occasionally. After centrifuging the mixture at 2,000 rpm at 4° C. for 12 min and confirming leucocytic pellet, a supernant was removed. If RBC (red blood cell) was left, said process was repeated. TRIZOL was added to finally obtain leucocytic pellet to separate RNA.

TABLE 1

| Cell types | | | | Cell collection centers |
|---|---|---|---|---|
| Culture | Cancer | A549 | Lung cell line | ATCC CCL-185 |
| cell lines | | HCT116 | Colon cancer cell | ATCC CCL-247 |
| | | Colo205 | line | ATCC CCL-222 |
| | | DLD-1 | | ATCC CCL-221 |
| | | HeLa | Cervical cancer | ATCC CCL-2 |
| | | C33A | cell line | ATCC HTB-231 |
| | | HT1080 | Breeding cancer | ATCC CCL-121 |
| | | | cell line | |
| | | AGS | Stomach cancer | ATCC CRL-1739 |
| | | YCC-2 | cell line | Cancer metastasis research center, College |
| | | | | of Medicine, Yonsei University |
| | | YCC-3 | | Cancer metastasis research center, College |
| | | | | of Medicine, Yonsei University |
| | | MDA-MB-231 | Breast cancer cell | ATCC HTB-26 |
| | | | line | |
| | | Caki-2 | Kidney cancer | Cancer metastasis research center, College |
| | | | cell line | of Medicine, Yonsei University |
| | | Capan-2 | Pancreas cancer | Cancer metastasis research center, College |
| | | | cell line | of Medicine, Yonsei University |
| | | HepG-2 | Hepatoma cell | ATCC HB-8065 |
| | | | line | |
| | | SK-Hep-1 | Liver cancer cell | ATCC HTB-52 |
| | | | line | |
| | | SK-Mel2 | Malignant | ATCC HTB-68 |
| | | | melanoma cell | |
| | | | line | |
| | | Jurket | Leukemia | Cancer metastasis research center, College |
| | | cDNA | | of Medicine, Yonsei University |
| | | library | | |
| | | U87-MG | Brian cancer cell | Korea cell line bank KCLB3004 |
| | | | line | |
| | Normal | 293 | Embryonic | Cancer metastasis research center, College |
| | | | kidney cell line | of Medicine, Yonsei University |
| Tissue | Cancer | SES-T | Intestine cancer | Cancer metastasis research center, College |
| | | | | of Medicine, Yonsei University |
| | | Hwang00 | Stomach cancer | Cancer metastasis research center, College |
| | | | | of Medicine, Yonsei University |
| | Normal | Human | Leucocyte | Cancer metastasis research center, College |
| | | Blood | | of Medicine, Yonsei University |
| | | SES-N | intestine | Cancer metastasis research center, College |
| | | | | of Medicine, Yonsei University |

### Example 2

### Preparation of mRNA and cDNA from Cell Lines

Total RNA was isolated from each tumor cell line, normal cell line and normal tissue using Trizol Reagent (Gibco-BRL, USA). 1 ml of Trizol Reagent was added to a tissue sample ground after quickly freezing using liquid nitrogen, followed by incubation at room temperature for 5 min. 0.2 ml of chloroform was added to the resulting tissue sample, vigorously vortexed for 15 sec and incubated at room temperature for 5 min. After centrifugation at 12,000×g at 4° C. for 15 min, the resultant aqueous phase was transferred to a new tube. An equal volume of isopropanol was added to the tube, and the tube was placed at 4° for 10 min. After centrifugation at 12,000×g at 4° C. for 10 min, the supernatant was carefully discarded, and the pellet was washed with 70% ethanol, followed by centrifugation at 7,500×g at 4° C. for 5 min. After being dried, the RNA pellet was dissolved in RNase-free water.

To synthesize cDNA from mRNA isolated from each cell line, and human-derived tumor and normal cell line, RT-PCR was performed as follows. 2 μg of total RNA was mixed with 1 μL of an oligo(dT)₁₆-primer, and RNase-free water was added up to a final volume of 11 μL. This mixture was heated at 90° C. for 5 min, and placed on ice, immediately after completion of the heating. After putting 4 μL of a reaction buffer, 2 μL of 10 mM dNTPs, 1 μL of RNase inhibitor and 2 μL of reverse transcriptase into another tube, 8.5 μL of the RNA mixture was added to the pre-mixture tube, followed by incubation at room temperature for 10 min. The reaction mixture was incubated at 42° C. for 90 min, and then at 95° C. for 15 min. Immediately after the incubation at 95° C., the mixture was placed on ice to terminate reaction, thus yielding a cDNA sample.

### Example 3

### Preparation of DNA Chip 1 for Examining Effectiveness of Probe for Cancer Diagnosis

In order to investigate whether a DNA chip can be used as a tool for detection of a splice junction site of G-CSF mRNA or cDNA, various DNA fragment probes capable of being immobilized on a glass plate was prepared as follows. On probe corresponding to a part of exon 2 of G-CSF, four non-overlapping probes corresponding to exon 3, and one probe corresponding to a part of exon 4, were designed to consist of 20 nucleotides each. Since two different G-CSF mRNAs (human G-CSFa and human G-CSFb mRNAs) are generated by alternative splicing in the exon 2 region (Tshuchiya, M. et al., EMBO J., 5:575˜581, 1986), two types of probes comprising a region corresponding to exon 2 were prepared, based on the two different G-CSF mRNAs. Nucleic acid sequences thereof are shown in Table 2.

TABLE 2

| Probe | | SEQ ID | |
|---|---|---|---|
| name | Nucleic acid sequences | NO: | Positions |
| E2 | CTG CAG CTG CTG CTG TGG CAC | 3 | Exon 2 |
| E2E3a | AGA AGC TGT GTG CCA C | 4 | Exon 2-3 |
| E2E3b | TGA GTG AGT GTG CCA C | 5 | Exon 2-3 |
| E3-1 | TGT GCC ACC TAC AAG CTG TG | 6 | Exon 3 |
| E3-3 | GAG CTG GTG ATG CTC GGA | 7 | Exon 3 |
| E3-4 | GGA CAC TCT CTG GGC ATC | 8 | Exon 3 |
| E3-6 | GGA CAC TCT CTG GGC ATC | 9 | Exon 3 |
| E4 | GCA GGC TGC TTG AGC CAA | 10 | Exon 4 |
| E2E4a | AGA AGC TGG CAG GCT G | 11 | Exon 2-4 |
| E2E4b | TGA GTG AGG CAG GCT G | 12 | Exon 2-4 |

To confer ability to be immobilized on a glass plate, when synthesizing all DNA fragment probes, a base having an amino group was inserted to the 3′-end of the probes using an aminolinker column (Cruachem, Glasgrow, Scotland), and slide glass coated with aldehyde residues (CEL Associates, Inc., Houston Tex., USA) were used.

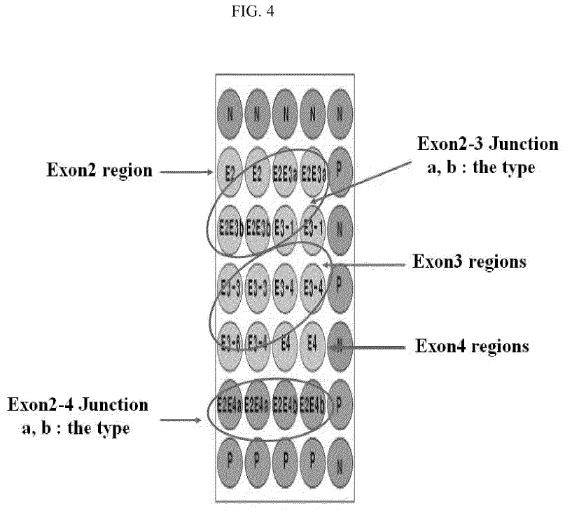

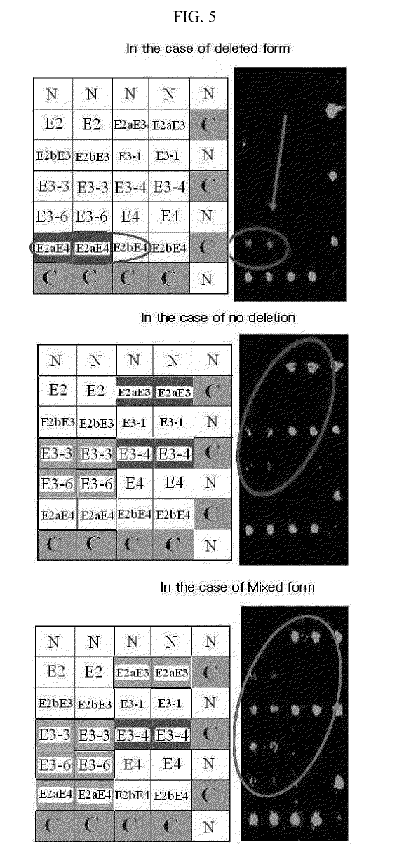

After being dissolved in 3×SSC (0.45 M NaCl, 15 mM C₆H₅Na₃O₇, pH 7.0), the DNA probes were immobilized on the slide glass by accumulating the DNA probes using a microarrayer manufactured by the present inventors (Yoon et al., J. Microbiol. Biotechnol., 10:21˜26, 2000), and reacting for over 1 hr under about 55% humidity, and then leaving the glass at room temperature for 6 hrs (FIG. 4). Herein, the probes were arranged at intervals of 180 μm on the glass at an amount of 100 μM, thus producing a microarray. Immobilization of probes through reaction between amine groups of probes and aldehyde groups on the glasses was estimated by staining with SYBRO green II (Molecular Probes, Inc., Leiden, Netherlands).

### Example 4

### Preparation of Target Sample for Detecting Specific Variants

Asymmetric PCR was carried out using mRNA or cDNA isolated from each cell line of Example 2 as a template under the conditions of denaturation at 94° C. for 5 min, 30 cycles of denaturation at 94° C. for 1 min, annealing at 50˜56° C. for 1 min and extension at 72° C. for 30 sec, followed by final extension at 72° C. for 5 min. A primer set used in Asymmetric PCR is as follows. A reverse primer was labeled with FITC for detection.

(SEQ ID NO: 13)

Forward primer: 5′-ACC CCC CTG GGC CCT GCC-3′

(SEQ ID NO: 14)

Reverse primer: FITC-5′CTG CTG CCA GAT GGT GGT-3′

PCR products were separated on an agarose gel. From the result of electrophoresis, double strand DNA and single stranded DNA fragments were produced in each PCR sample (FIG. 3). After amplifying G-CSF gene by asymmetric PCR, a hybridization solution (6×SSPE, 20% (v/v) foramide) was added to 15 μL of the amplified product up to a final volume of 200 μL. The mixture was applied on a slide glass (a DNA chip 1 for cancer diagnosis, FIG. 4) having an immobilized probe, and the glass was covered with a probe-clip press seal incubation chamber (Sigma Co., St. Louis, Mo.), followed by incubation in a shaking incubator at 30° C. for 6 hours to induce binding of the probe complemantary to the amplified product. Thereafter, the glass was washed over 5 min with 3×SSPE (0.45 M NaCl, 15 mM C₆H₅Na₃O₇, pH7.0), 2×SSPE (0.3M NaCl, 10 mM C₆H₅Na₃O₇, pH7.0), and then 1×SSPE (0.15 M NaCl, 5 mM C₆H₅Na₃O₇, pH7.0).

### Example 5

### Test Result of DNA Chip 1 for Cancer Diagnosis

After target products amplified by Asymmetric PCR was applied to the DNA chip prepared in Example 3, they were scanned using Scanarray 5000 (GSI Lumonics Inc., Bedford, Mass., USA). To predict results regarding probe, in case of the plasmid having no deletion of exon 3 in G-CSF gene, signals were detected by applying on the DNA chip. In contrast, in case of the exon 3-deleted G-CSF-containing plasmid, signals were detected by applying on the DNA chip, wherein the plasmids have nucleotide sequences of SEQ ID NOs: 26 and 27.

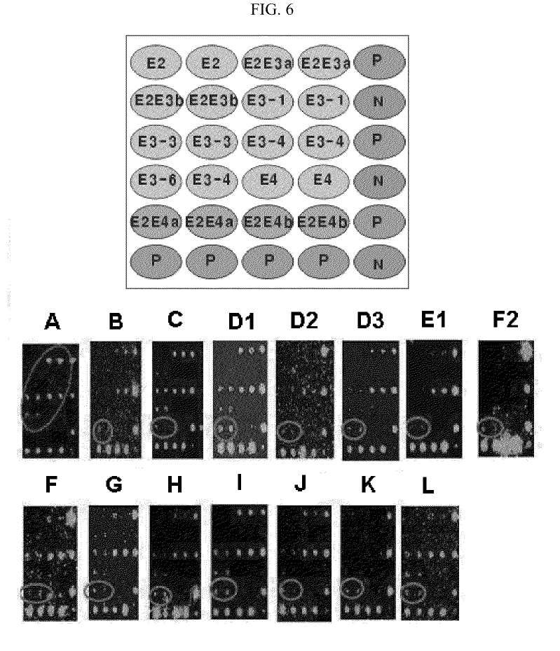

As a result, as shown in FIG. 5, only E2E4a probe showed signals on a deletion site. This plasmid had type A exon 2 (FIG. 1). On the contrary, in the case of plasmid in which G-CSF gene was not deleted, E2E3a probe and probes in exon 3 region showed signals. If the sequences of no deletion and of deletion of exon 3 in G-CSF were mixed, mixed results of the two cases can be predicted. FIG. 6 shows the hybridization results by Scanarray 5000 after target DNA according to each cell was applied to DNA chip of FIG. 4. As shown in FIG. 6, in case where probes produced from exon 2 and exon 4 junction region, which only specific variants can have, cells could be detected by each probe.

### Example 6

### Preparation of DNA Chip 2 for Examining Effectiveness of Probe for Cancer Diagnosis and Test Results

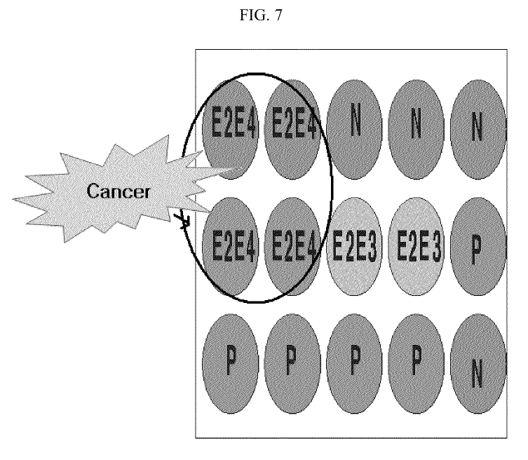

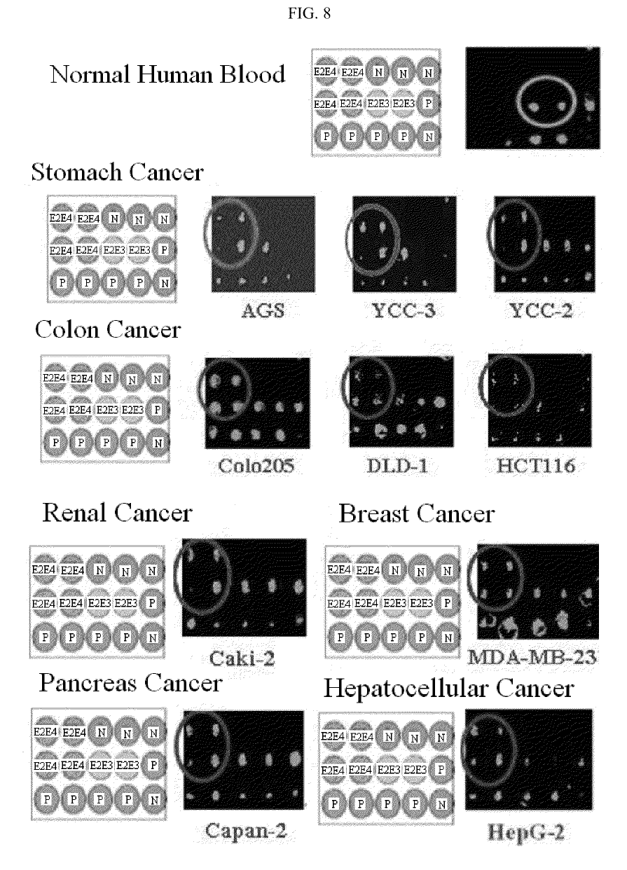

To examine effectiveness of E2E4 for cancer diagnosis, a new type DNA chip 2 was prepared (FIG. 7). To easily decode, the DNA chip 2 was designed to have two types of exons (E2E4 of FIG. 7 contains both type A and type B, E2E3 contains both type A and type B of E2E3a and E2E3b). Probes were immobilized by the same immobilization method described in Example 3, and as a result of hybridization of a target sample prepared in Example 4, as shown in FIG. 8, it was confirmed that probes constructed from the junction region of exon 2 and exon 4 is the most powerful in developing a system which can easily diagnose cancer using produced DNA chip. To strengthen signal intensity, probes having nucleic acid sequences in Table 3 below were applied on the basis of a nucleic acid sequence of splice junction site.

TABLE 3

| Probe | | SEQ ID | |
|---|---|---|---|
| name | Nucleic acid sequences | NO: | Positions |
| E2E4a | GGA GAA GCT GGC AGG CTG CT | 1 | Exon 2-4 |
| E2E4b | GGT GAG TGA GGC AGG CTG CT | 2 | Exon 2-4 |

### Example 7

### Preparation of DNA Chip 3 for Examining Effectiveness of Probe for Cancer Diagnosis

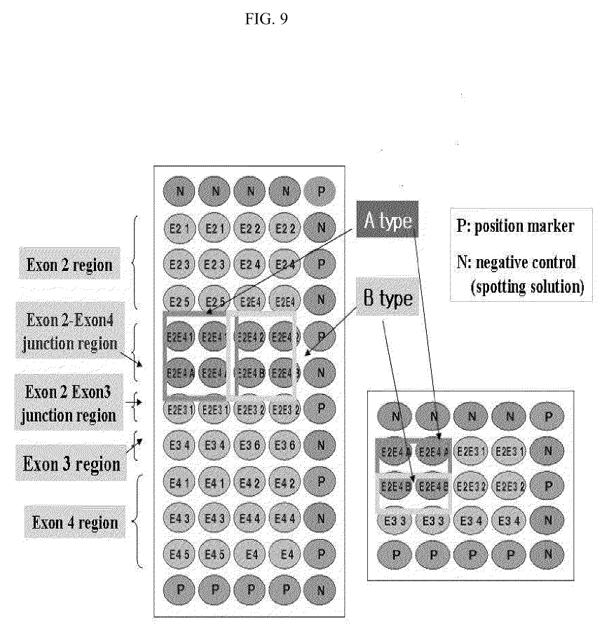

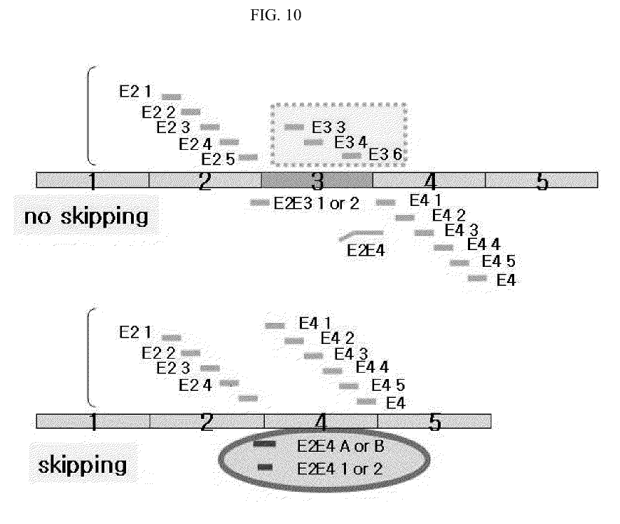

To examine whether probes constructed from exon 2 and exon 4 junction region were the most powerful; DNA chip 3 was prepared by designing probes from each nucleotide sequence in each region (FIG. 9). FIG. 10 shows the rough position of each probe in G-CSF gene, and Table 4 shows nucleic acid sequence of each probe. Probes were fixed by the same immobilization method described in Example 3.

TABLE 4

| Probe | | SEQ ID | |
|---|---|---|---|
| name | Nucleic acid sequences | NO: | Positions |
| E2 1 | GAG CTT CCT GCT CAA GTG CT | 15 | Exon 2 |
| E2 2 | AGA GCT TCC TGC TCA AGT GC | 16 | Exon 2 |
| E2 3 | GCA AGT GAG GAA GAT CCA GG | 17 | Exon 2 |
| E2 4 | CCA GAG CTT CCT GCT CAA GT | 18 | Exon 2 |
| E2 5 | CAA GTG AGG AAG ATC CAG GG | 19 | Exon 2 |
| E2E4 | CTG GTG AGT GGC AGG CTG CT | 20 | Exon 2-3-4 |
| E2E4 1 | AGA AGC TGG CAG GCT G | 9 | Exon 2-4 |
| E2E4 2 | TGA GTG AGG CAG GCT G | 10 | Exon 2-4 |
| E2E4a | GGA GAA GCT GGC AGG CTG CT | 13 | Exon 2-4 |
| E2E4b | GGT GAG TGA GGC AGG CTG CT | 14 | Exon 2-4 |
| E2E3 1 | AGA AGC TGT GTG CCA A | 2 | Exon 2-3 |
| E2E3 2 | TGA GTG AGT GTG CCA C | 3 | Exon 2-3 |
| E3 3 | GAG CTG GTG CTG CTC GGA | 5 | Exon 3 |
| E3 4 | GGA CAC TCT CTG GGC ATC | 6 | Exon 3 |
| E3 6 | GGA CAC TCT CTG GGC ATC | 7 | Exon 3 |
| E4 1 | CTT TTC CTC TAC CAG GGG CT | 21 | Exon 4 |
| E4 2 | CAT AGC GGC CTT TTC CTC TA | 22 | Exon 4 |
| E4 3 | TTT TCC TCT ACC AGG GGC TC | 23 | Exon 4 |
| E4 4 | TAG CGG CCT TTT CCT CTA CC | 24 | Exon 4 |
| E4 5 | CGG CCT TTT CCT CTA CCA G | 25 | Exon 4 |
| E4 | GCA GGC TGC TTG AGCC CAA | 8 | Exon 4 |

### Example 8

### Test of DNA Chip 3 for Examining Effectiveness of Probe for Cancer Diagnosis

After target products amplified by Asymmetric PCR described in Example 4 was applied to the DNA chip 3 prepared in Example 7 (FIG. 9), they were scanned using Scanarray 5000 (GSI Lumonics Inc., Bedford, Mass., USA). In advance, the chip signals were tested both in case of using the sequence having no deletion of exon 3 and in case of using the sequence not having exon 3 in G-CSF gene by applying on the DNA chip.

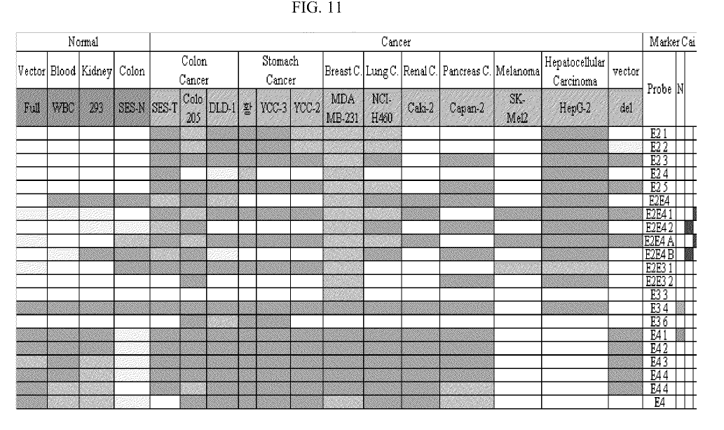

FIG. 11 shows the results of each probe according to applied biological samples. Samples marked with green in the left side of Table show the results of samples classified as normal, samples marked with red in the middle show the results of samples classified as cancer. The right side represents the results on final candidates of diagnostic cancer markers by analyzing all the results thereof. The degree of yellow in each column of Table shows the presence of a signal and intensity thereof, and red colors in column of the right side of Table represent strong probe candidates having effectiveness, which can detect cancer.

As shown in FIG. 11, probes constructed from exon 2 and exon 4 junction region are a powerful probe which can detect cancer among probe candidates which are designed from each exon region. Herein, a probe for type A shows signals with high intensity in most case of cancer and in case where signals are detected on SEQ ID NO: 4 and SEQ ID NO: 1 simultaneously, it could be interpreted that the probe has cancer-specific variants having exon 2 of type A. In the same line, in case where signals are detected on SEQ ID NO: 5 and SEQ ID NO: 2 simultaneously, it could be interpreted that the probe has cancer-specific variants having exon 2 of type B (FIG. 1).

On the contrary, powerful candidates which can distinguish normal cells is a probe (SEQ ID NO: 8) from exon 3 region, however because signals are shown in almost all samples among cancer samples to which this probe is applied, distinguishing between normal samples and cancer samples is impossible using the existence of this probe. Also, because probes from different site of exon 3 region doesn't show signals in both normal sample and cancer sample due to their weak sensitivity, distinguishing between normal sample and cancer sample is impossible using the probes.

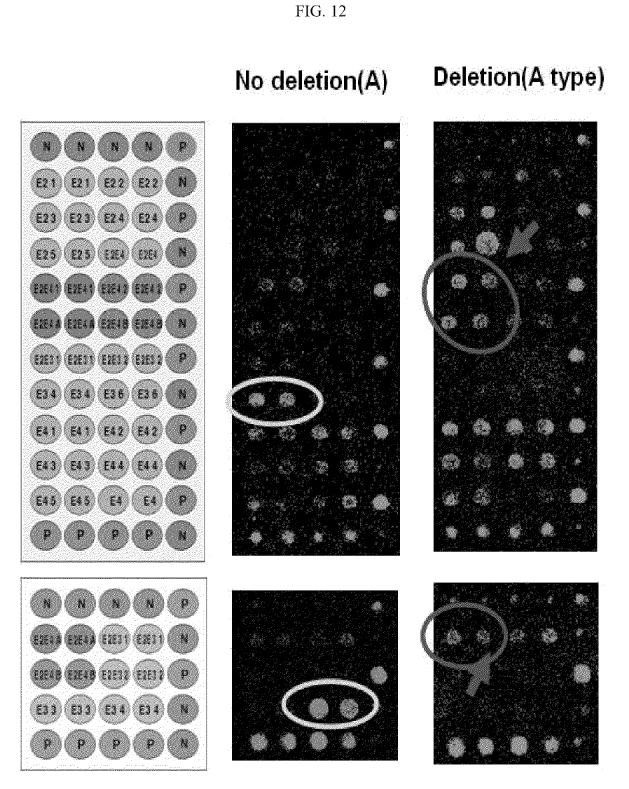

Target sample was amplified by Asymmetric PCR using a plasmid having exon 2 region of type A as a template as described in Example 4 and the target sample was applied to DNA chip 3 (FIG. 9). As a result, as shown in FIG. 12, E2E4a probe only showed signals in a plasmid sample in which exon 3 of G-CSF gene was deleted. Nucleotide sequences of plasmids having no deletion of G-CSF gene and deletion of G-CSF gene are SEQ ID NO: 26 and SEQ ID NO: 27, respectively.

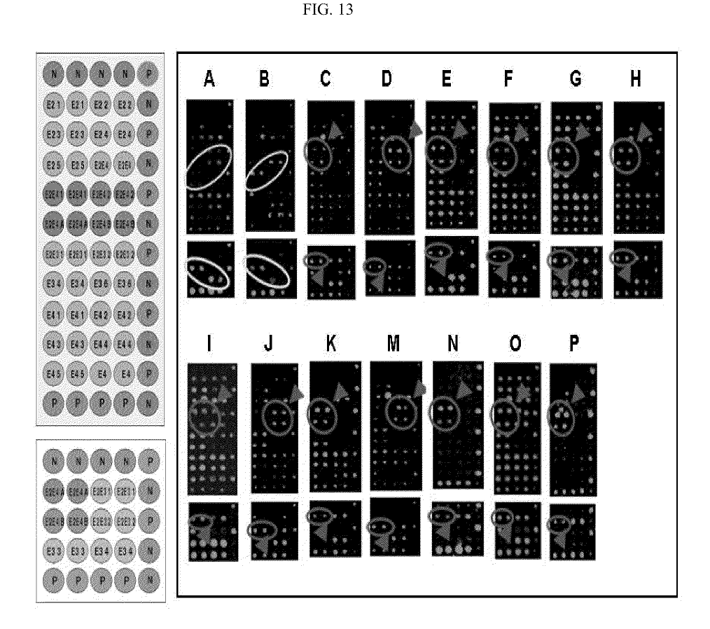

FIG. 13 shows the results of detection by Scanarray 5000 after target DNA according to each sample was applied to DNA chip 3 of FIG. 11. As shown in FIG. 13, it was confirmed that the existence of cancer could be detected by existence of signals on the probes constructed from splice junction site of exon 2 region and exon 4 region, which is the only basis of distinguishing cancer cells by each probe. Sites marked with red circles are diagnostic cancer markers whose effectiveness was confirmed by the present inventors.

### Example 9

### Isolation of RNA from Blood or Tissues of Normal Individuals and Patients

Total RNA from each cancer cell lines, normal blood and normal tissues was isolated using TRIZOL® REAGENT (GIBCO-BRL, USA). In case of blood, it was isolated using TRIZOL® LS REAGENT (GIBCO-BRL, USA). To prepare a sample, blood and LS REAGENT are added in a ratio of 1:3. According to case, blood sample was previously diluted in a ratio of 1:1, then REAGENT can be added in a ratio of 1:3. 0.75 mL of TRIZOL LS Reagent was added to 0.25 mL of blood sample (or diluted blood sample) and RNA can be extracted according to protocol. In case of tissues, 1 mL of Trizol reagent was added to a tissue sample ground after quickly freezing using liquid nitrogen to isolate RNA according to protocol.

The resulting tissue sample added with 1 mL of Trizol Reagent was incubated at room temperature for 5 min. The resulting tissue sample was supplemented with 0.2 mL of chloroform, vigorously mixed for 15 sec, and incubated at room temperature for 5 min. After centrifugation at 12,000×g at 4° C. for 15 min, the resultant aqueous phase was transferred to a new tube. An equal volume of isopropanol was added to the tube, and the tube was placed at 4° C. for 10 min. After centrifugation at 12,000×g at 4° C. for 10 min, the supernatant was carefully discarded, and the pellet was washed with 70% ethanol, followed by centrifugation at 7,500×g at 4° C. for 15 min. After being dried, the RNA pellet was dissolved in RNase-free water.

### Example 10

### Amplification of G-CSF Gene from RNA

To synthesize cDNA from mRNA isolated from each cell line, and human-derived tumor and normal cell line and to amplity G-CSF gene, RT-PCR was performed as follows. 1˜2 μL of total RNA and 8 μL of ONE-STEP PCR premix (Intron Inc., Korea) were mixed with primers of SEQ ID NOs: 28 and 29 in Table 5, and RNase-free water was added up to a final volume of 20 μL. Then, G-CSF gene can be directly amplified from RNA by carrying out an amplification reaction under the condition described in Table 5. GAPDH was amplified using primers of SEQ ID NO: 30 and SEQ ID NO: 31 and it was used as a control for RNA amplification.

TABLE 5

| SEQ ID | | | Nucleic acid sequences |
|---|---|---|---|
| NO: | Primer | Name | (5′→3′) |
| 28 | 1 | Ex1-Fw | AGA GCC CCA TGA AGC TGA T |
| 29 | 2 | ex5-Re | GAC ACC TCC AGG AAG CTC TG |
| 30 | 3 | GAPDH F | CAT CTT CCA GGA GCG AGA CC |
| 31 | 4 | GAPDH R | TCC ACC ACC CTG TTG CTG TA |
| 32 | 5 | Full F | ACC CCC CTG GGC CCT GCC |
| 33 | 6 | E4fullRe | CTG CTG CCA GAT GGT GGT |

TABLE 6
One Cycle

| | |
|---|---|
| Reverse transcription reaction | 45°/30 min |
| Non-activation of RTase | 90°/5 min |

3-Step-Cycling

| | |
|---|---|
| Denaturation | 94°/20-60 sec |
| Annealing | 50°/20-60 sec |
| Extension | 72°/1 min/kb |
| Number of cycles: 35 | |

One Cycle

| | |
|---|---|
| Final extension | 72°/5 min |

hG-CSF was amplified using 1˜2 μL of first PCR product as template, which was amplified by ONE-STEP PCR method (Table 2), based on 50 μL of total reaction volume with primers of SEQ ID NO: 32 and SEQ ID NO: 33, wherein SEQ ID NO: 33 was labeled with fluorescence (Cy5 or different kind of fluorescence). Asymmetric PCR which has a big difference in addition ratio of forward primer (SEQ ID NO: 32) and reverse primer (SEQ ID NO: 33) from 1:5 to 1:10 was secondarily performed to obtain final amplification products.

GAPDH can be also obtained by labeling reverse primer (SEQ ID NO: 31) with fluorescence to perform an amplification reaction, as described the above.

### Example 11

### Preparation of DNA Chip for Applying to Diagnosis of Patients and Hybridization Results

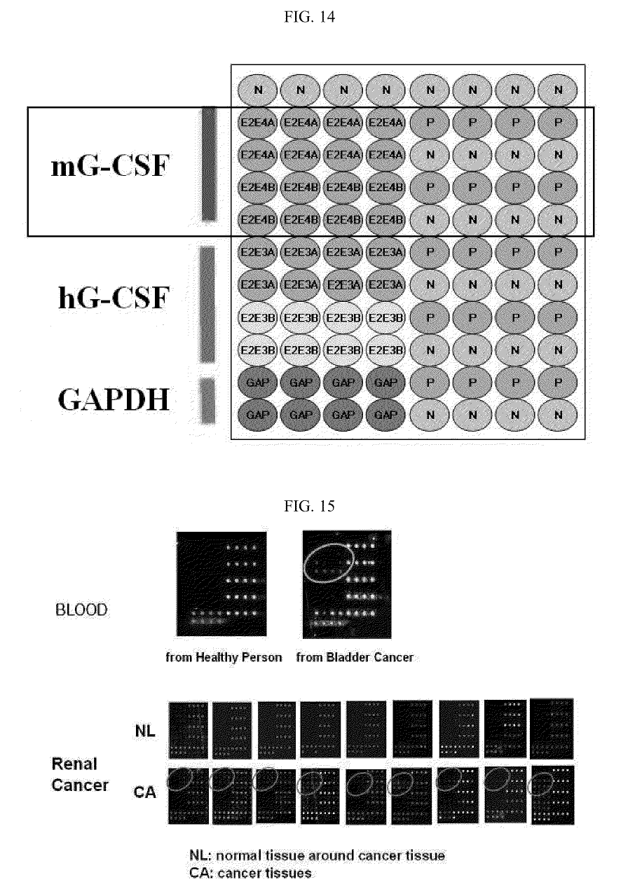

DNA chip was prepared by mixing each probe (E2E4a and E2E4b) in 3×SSC spotting solution at a concentration of 50 μM (FIG. 14). The part marked with a blue square in FIG. 14 is the region on which probes indicating cancer are located.

PCR products from normal individuals and patients amplified using primers of SEQ ID NO: 32 and SEQ ID NO: 33 were hybridized to the DNA chip according to Example 8 and Example 9 (FIG. 15). To identify a control for the experiment, GAPDH amplified using primers of SEQ ID NO: 30 and SEQ ID NO: 31 were also hybridized. Applied patients were shown in each figure. In FIG. 15, purple ellipticals show signals in probes indicating cancer.

As shown in FIG. 15, as a result of application of DNA chip for cancer diagnosis according to the present invention to diagnosis of patients, it was confirmed that probes for cancer diagnosis according to the present invention are excellent as a diagnostic cancer marker on the prepared DNA chip.

### INDUSTRIAL APPLICABILITY

As described and proven above in detail, the present invention provides an oligonucleotide essentially containing a nucleic acid sequence of a splice junction site having the 3′-terminal end of exon 2 region linked to the 5′-terminal end of exon 4 region of a G-CSF gene, a diagnostic kit for cancer diagnosis containing the oligonucleotide and a method for diagnosing cancer using the nucleic acid molecule. According to the present invention, cancer can be quickly and exactly diagnosed using variation of a G-CSF gene.

Although a specific embodiment of the present invention has been described in detail, those skilled in the art will appreciate that this description is merely a preferred embodiment and is not construed to limit the scope of the present invention. Thus, the substantial scope of the present invention will be defined by the accompanying claims and equivalents thereof.

1. An oligonucleotide for diagnosing cancer, essentially containing a nucleic acid sequence of a splice junction site having the 3′-terminal end of exon 2 region linked to the 5′-terminal end of exon 4 region of a granulocyte colony stimulating factor gene.

2. The oligonucleotide for diagnosing cancer according to claim 1, which essentially contains nucleic acid sequences of SEQ ID NOs: 1 or 2.

3. A diagnostic kit for cancer diagnosis, containing the oligonucleotide of claim 1.

4. The diagnostic kit for cancer diagnosis according to claim 4, wherein said diagnostic kit assesses the state of cancer progression and additionally contains an oligonucleotide essentially containing sequences of a part or the entire region of the exon 3 region of G-CSF gene.

5. The diagnostic kit for cancer diagnosis according to claim 4, wherein said oligonucleotide essentially contains nucleic acid sequences of SEQ ID NOs: 1 or 2.

6. The diagnostic kit for cancer diagnosis according to claim 3, wherein said kit is microarray.

7. A method for diagnosing cancer, the method comprising the steps of:
(a) obtaining a G-CSF nucleic acid sample from a mammal biological sample;
(b) amplifying the obtained G-CSF nucleic acid sample; and
(c) detecting oligonucleotide containing a nucleic acid sequence of a splice junction site having the 3′-terminal end of exon 2 region linked to the 5′-terminal end of exon 4 region of a G-CSF gene, in the amplifying sample.

8. The method for diagnosing cancer according to claim 7, wherein the step (c) simultaneously detects an oligonucleotide containing sequences of a part or the entire region of the exon 3 region together with an oligonucleotide containing a nucleic acid sequence of splice junction site having the 3′-terminal end of exon 2 region linked to the 5′-terminal end of exon 4 region of a G-CSF gene, in the amplifying sample.

9. The method for diagnosing cancer according to claim 7, wherein said oligonucleotide essentially contains nucleic acid sequences of SEQ ID NOs: 1 or 2.
